(19) United States
(12) Patent Application Publication
(10) Pub. No.: US 2024/0269436 A1
(43) Pub. Date: Aug. 15, 2024

(54) CATHETER INSERTION DEVICE

(71) Applicant: Becton, Dickinson and Company, Franklin Lakes, NJ (US)

(72) Inventor: Scott STEWART, Somerville, MA (US)

(73) Assignee: Becton, Dickinson and Company, Franklin Lakes, NJ (US)

(21) Appl. No.: 18/563,257

(22) PCT Filed: May 26, 2022
(86) PCT No.: PCT/US2022/031013

§ 371 Date: Nov. 21, 2023

Related U.S. Application Data
(60) Provisional application No. 63/193,141, filed on May 26, 2021.

(51) Int. Cl. A61M 25/06; A61M 5/142; A61M 5/42

(52) U.S. Cl. CPC A61M 25/0606; A61M 5/427; A61M 2005/14252

(57) ABSTRACT

A catheter insertion device (10) includes a base (12) configured for contacting a skin surface of a patient, a catheter hub (14) having a catheter (16) and a needle carrier (18) having an insertion needle (20). The catheter hub (14) is movable with respect to the base. The catheter is coupled to the catheter hub and extends distally from the base and the catheter hub. The insertion needle extends through the catheter hub and the catheter. The needle carrier is coupled to the catheter hub and movable with respect to the base. The needle carrier is configured to position the catheter hub in a first position with respect to the base during insertion of the catheter in the patient, and to move the catheter hub and catheter to a second position with respect to the base when the insertion needle is retracted.

This application is a U.S. national stage application of International Patent Application No. PCT/US2022/031013, filed May 26, 2022, which claims priority to US Provisional Patent Application No. 63/193,141, filed May 26, 2021, which is hereby incorporated by reference in its entirety.

### FIELD OF THE INVENTION

The present invention is directed to a cannula or catheter insertion device for use in a medication delivery device and to a method of inserting a cannula or catheter into a patient using the insertion device. The insertion device can be a catheter insertion device for use with a patch pump, infusion set or other delivery device for introducing a catheter into a patient and automatically retracting an insertion needle once the catheter is moved to an extended position. The insertion device is able to position the cannula or catheter into the patient with reduced tenting of the surface of the surface of the skin or tissue of the patient.

### BACKGROUND OF THE INVENTION

Diabetes is a group of diseases characterized by high levels of blood glucose resulting from the inability of diabetic patients to maintain proper levels of insulin production when required. Persons with diabetes will require some form of daily insulin therapy to maintain control of their glucose levels. Diabetes can be dangerous to the affected patient if it is not treated, and it can lead to serious health complications and premature death. However, such complications can be minimized by utilizing one or more treatment options to help control the diabetes and reduce the risk of complications.

The treatment options for diabetic patients include specialized diets, oral medications and/or insulin therapy. The main goal of diabetes treatment is to control the diabetic patient's blood glucose or sugar level. However, maintaining proper diabetes management may be complicated because it has to be balanced with the activities of the diabetic patient.

For the treatment of type 1 diabetes, there are two principal methods of daily insulin therapy. In the first method, diabetic patients use syringes or insulin pens to self-inject insulin when needed. This method requires a needle stick for each injection, and the diabetic patient may require three to four injections daily. The syringes and insulin pens that are used to inject insulin are relatively simple to use and cost effective.

Another effective method for insulin therapy and managing diabetes is infusion therapy or infusion pump therapy in which an insulin pump is used. The insulin pump can provide continuous infusion of insulin to a diabetic patient at varying rates in order to more closely match the functions and behavior of a properly operating pancreas of a non-diabetic person that produces the required insulin, and the insulin pump can help the diabetic patient maintain his/her blood glucose level within target ranges based on the diabetic patient's individual needs.

Infusion pump therapy requires an infusion cannula, typically in the form of an infusion needle or a flexible catheter, that pierces the diabetic patient's skin and through which, infusion of insulin takes place. Infusion pump therapy offers the advantages of continuous infusion of insulin, precision dosing, and programmable delivery schedules.

In infusion therapy, insulin doses are typically administered at a basal rate and in a bolus dose. When insulin is administered at a basal rate, insulin is delivered continuously over 24 hours in order to maintain the diabetic patient's blood glucose levels in a consistent range between meals and rest, typically at nighttime. Insulin pumps may also be capable of programming the basal rate of insulin to vary according to the different times of the day and night. In contrast, a bolus dose is typically administered when a diabetic patient consumes a meal, and generally provides a single additional insulin injection to balance the consumed carbohydrates. Insulin pumps may be configured to enable the diabetic patient to program the volume of the bolus dose in accordance with the size or type of the meal that is consumed by the diabetic patient. In addition, insulin pumps may also be configured to enable the diabetic patient to infuse a correctional or supplemental bolus dose of insulin to compensate for a low blood glucose level at the time when the diabetic patient is calculating the bolus dose for a particular meal that is to be consumed.

Insulin pumps advantageously deliver insulin over time rather than in single injections, typically resulting in less variation within the blood glucose range that is recommended. In addition, insulin pumps may reduce the number of needle sticks which the diabetic patient must endure, and improve diabetes management to enhance the diabetic patient's quality of life.

Typically, regardless of whether a diabetic patient uses multiple direct injections (MDIs) or a pump, the diabetic patient takes fasting blood glucose medication (FBGM) upon awakening from sleep, and also tests for glucose in the blood during or after each meal to determine whether a correction dose is required. In addition, the diabetic patient may test for glucose in the blood prior to sleeping to determine whether a correction dose is required, for instance, after eating a snack before sleeping.

To facilitate infusion therapy, there are generally two types of insulin pumps, namely, conventional pumps and patch pumps. Conventional pumps require the use of a disposable component, typically referred to as an infusion set, tubing set or pump set, which conveys the insulin from a reservoir within the pump into the skin of the user. The infusion set consists of a pump connector, a length of tubing, and a hub or base from which a cannula, in the form of a hollow metal infusion needle or flexible plastic catheter extends. The base typically has an adhesive that retains the base on the skin surface during use. The cannula can be inserted onto the skin manually or with the aid of a manual or automatic insertion device. The insertion device may be a separate unit required by the user.

Another type of insulin pump is a patch pump. Unlike a conventional infusion pump and infusion set combination, a patch pump is an integrated device that combines most or all of the fluidic components, including the fluid reservoir, pumping mechanism and mechanism for automatically inserting the cannula, in a single housing which is adhesively attached to an infusion site on the patient's skin, and does not require the use of a separate infusion or tubing set. A patch pump containing insulin adheres to the skin and delivers the insulin over a period of time via an integrated subcutaneous cannula. Some patch pumps may wirelessly communicate with a separate controller device (as in one device sold by Insulet Corporation under the brand name OmniPod®), while others are completely self-contained. Such devices are replaced on a frequent basis, such as every three days, when the insulin reservoir is exhausted or complications may otherwise occur, such as restriction in the cannula or the infusion site.

As patch pumps are designed to be a self-contained unit that is worn by the diabetic patient, it is preferable to be as small as possible so that it does not interfere with the activities of the user. Thus, in order to minimize discomfort to the user, it would be preferable to minimize the overall thickness of the patch pump. However, in order to minimize the thickness of the patch pump, its constituent parts should be reduced as much as possible. One such part is the insertion mechanism for automatically inserting the cannula into the user's skin.

Although the prior devices achieve the intended purpose, there is need for an improved insertion mechanism for introducing the cannula or catheter into the patient for controlling the depth of penetration.

### SUMMARY OF THE INVENTION

The present invention is directed to a cannula or catheter insertion device that can be used with an infusion set or patch pump and to a method of deploying a catheter and/or inserting a catheter into a patient using the insertion device. The invention in one embodiment is a catheter insertion device that is able to position the cannula or catheter at a selected depth in the skin of the patient. The cannula or catheter can be used in conjunction with the delivery of a medication to a patient. The insertion device is mainly directed to a device for insulin delivery by an infusion set.

One feature of the insertion device is having a manually operated device that is pressed by the user to deploy the cannula or catheter. The device is able to separate from the cannula or catheter and to retract an introducer needle. In one embodiment, the device is primarily for inserting the cannula or catheter into a patient.

A feature of the insertion device is able to introduce a cannula or catheter into surface of a substrate to a selected depth. The device is configured for introducing the cannula or catheter into a patient for delivering a medication while reducing tenting of the surface of the skin of the patient at the insertion site to control the depth of penetration of the catheter in the skin of the patient and for controlling the location of delivery of the medication into the patient. Reducing the tenting of the surface of the skin at the insertion site can be controlled by inserting the insertion needle and catheter into the skin at a first depth and withdrawing the catheter a selected amount to reduce the tenting and to position the distal end of the catheter at a depth and location in the patient for the selected delivery of the medication.

In one embodiment, the insertion device has a manually operated needle carrier of an insertion device to contact a catheter hub assembly to insert the catheter into the patient. The needle carrier is able to retract the catheter a limited distance with respect to a base of the insertion device before withdrawing the needle from the catheter.

The insertion device can include a base with a movable catheter hub where the catheter hub can be inserted a first depth during the initial insertion and retracted a selected amount to position the distal end of the catheter from a bottom surface of the base a selected amount. By retracting the catheter hub and the catheter a selected distance, the tenting of the skin surface at the insertion site of the patient can be reduced. An insertion needle can be withdrawn from the catheter and the catheter hub can then be connected to a delivery device.

The features of the insertion device are basically attained by providing an insertion device having a base, a catheter hub movable between a first extended position and a second retracted position with respect to the base, an introducer needle within the catheter and needle carrier movable with respect to the base to move the catheter hub from the first extended position to the second retracted position.

The various aspects of the invention are also attained by providing a catheter insertion device comprising a base configured for contacting a skin surface of a patient, a catheter hub movable relative to the base, a catheter coupled to the base, and a needle carrier. The catheter extends from a distal side of the catheter hub and from a distal side of the base. The needle carrier has an insertion needle extending through the catheter. The needle carrier is coupled to the catheter hub and is movable with respect to the base. The needle carrier is configured to position the catheter hub in a first position with respect to the base during insertion of the catheter and insertion needle into the patient and to move the catheter hub and catheter to a second position with respect to the base when the insertion needle is retracted.

The features of the catheter insertion are also provided by a method of deploying a catheter. The method can used for introducing a catheter into a surface, such as a patient, the method including the steps of introducing a catheter and an insertion needle into a skin surface of the patient to a first depth, retracting the catheter and insertion needle to a second depth to reduce tenting of the skin surface, where the second depth in less than the first depth. The insertion needle is then retracted from the catheter hub and the catheter where the catheter can be connected to a medication source for introducing the medication to the patient.

These and other aspects of the invention will become apparent from the following detailed description of the invention which, taken in conjunction with the annexed drawings, show various embodiments of the insertion device.

### BRIEF DESCRIPTION OF THE DRAWINGS

The various objects, advantages and novel features of the exemplary embodiments of the present invention will be more readily appreciated from the following detailed description when read in conjunction with the appended drawings, in which:

FIG. 1 is a perspective view of one embodiment of the insertion device;

FIG. 2 is an exploded view of the insertion device of FIG. 1;

FIG. 3 is a cross sectional view of the insertion device during insertion of the catheter into the surface;

FIG. 4 is a cross sectional view of the insertion device of FIG. 1 after the initial insertion;

FIG. 5 is a schematic view of the catheter and needle insertion;

FIG. 6 is a cross sectional view of the insertion device in another embodiment;

FIG. 7 is an exploded view of the insertion device of FIG. 6;

FIG. 8 is a cross-sectional view showing the insertion device after retracting the catheter;

FIG. 9 is an elevational view of insertion device in a further embodiment;

FIG. 10 is a cross-sectional view showing the insertion device with the insertion needle in the extended position and the catheter inserted into the patient;

FIG. 11 is a cross sectional view of the insertion device partially withdrawn from the patient; and

FIG. 12 is a cross-sectional view showing the carrier separated from the catheter hub.

### DETAILED DESCRIPTION OF THE EXEMPLARY EMBODIMENTS

Reference is made in detail to embodiments of the present invention, which are illustrated in the accompanying drawings, wherein like reference numerals refer to like elements throughout. The embodiments described herein exemplify, but do not limit, the present invention by referring to the drawings.

It will be understood by one skilled in the art that this disclosure is not limited in its application to the details of construction and the arrangement of components set forth in the following description or illustrated in the drawings. The embodiments herein are capable of being practiced or carried out in various ways. Also, it will be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless limited otherwise, the terms “connected,” “coupled,” and “mounted,” and variations thereof herein are used broadly and encompass direct and indirect connections, couplings, and mountings. In addition, the terms “connected” and “coupled” and variations thereof are not restricted to physical or mechanical connections or couplings. Further, terms such as up, down, bottom, and top are relative, and are employed to aid illustration, but are not limiting. Any of the embodiments and/or elements and features disclosed herein may be combined with one another to form various additional embodiments not specifically disclosed, as long as they do not contradict or are not inconsistent with each other. Terms of degree, such as “substantially”, “about” and “approximately” are understood by those skilled in the art to refer to reasonable ranges around and including the given value and ranges outside the given value, for example, general tolerances associated with manufacturing, assembly, and use of the embodiments. The term “substantially” when referring to a structure or characteristic includes the characteristic that is mostly or entirely.

The device and method of use are broadly directed to deploying a catheter or cannula and to inserting or positioning the catheter or cannula into surface of a substrate. The illustrative embodiments are described with reference to injecting a medication into a patient at a selected depth. It is to be understood that these illustrative embodiments can be used with different drug therapies and regimens to treat physiological conditions other than diabetes using different medicaments. In the embodiment described, the device is configured for insulin delivery using a cannula or catheter although it is understood that the disclosure is not limited to a particular delivery device.

The device as described herein include an insertion device for assisting in introducing a medication into a patient in a controlled manner and to a controlled depth within the skin surface of a patient. The insertion device is configured for reducing the tenting of the skin surface caused by the insertion of a cannula or catheter into the skin surface. The insertion device as described herein inserts the catheter and insertion needle into the skin surface, which can cause tenting of the skin surface by the catheter and insertion needle and the drag on skin surface. The device is configured to retract the insertion needle and catheter a predetermined distance to allow the skin surface to relax, return to its original configuration and reduce tenting on the surface of the skin at the insertion site. Reducing the tenting on the skin surface provides a controlled depth of penetration and controlled depth of delivery of the medication.

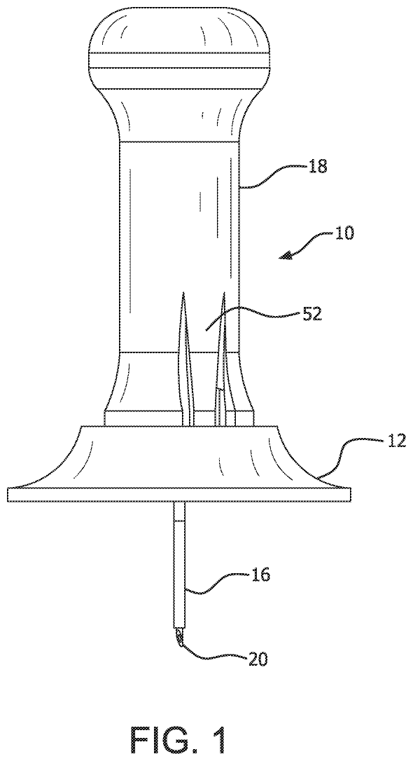

Referring to FIGS. 1-4, a first embodiment of the insertion device is shown. The insertion device 10 in the embodiment shown is an insertion device for deploying a catheter. The device 10 is suitable for inserting the catheter into a skin surface for the delivery of a medication, such as insulin. The insertion device 10 includes a base 12, a catheter hub 14, a catheter 16, a needle carrier 18 having an insertion needle 20.

The insertion device 10 as shown and described is configured primarily for use with a medication delivery device and particularly an insulin delivery device. The delivery device is not shown for clarity but can be any known suitable delivery device as known in the art that can connect to a catheter hub of catheter device for supplying the medication. The delivery device can be an insulin infusion pump, a patch pump or other delivery device that can provide a sustained delivery of the insulin the patient.

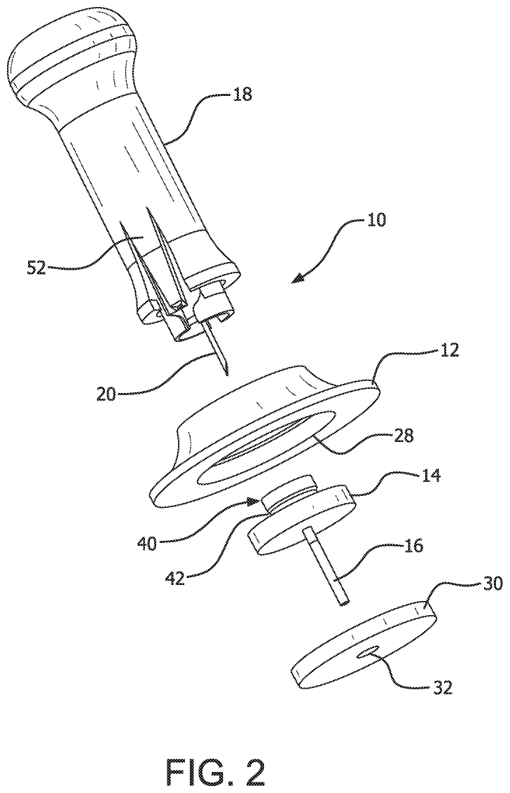

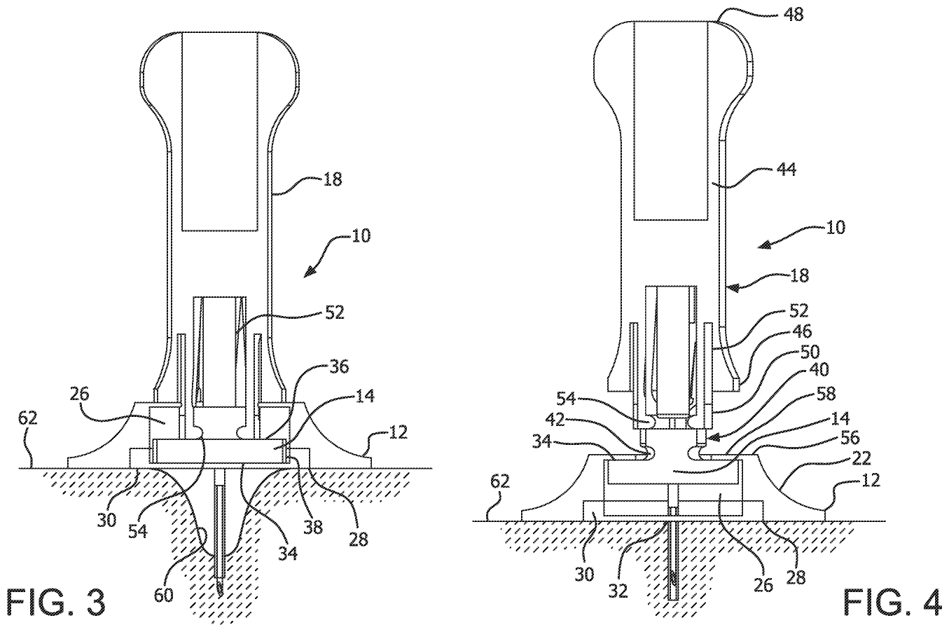

The base 12 is configured for supporting the catheter hub 14 and catheter 16 for introducing the medication to the patient. The base 12 has body 22 with a distal surface 24 configured to contact a surface, such as the skin surface by an adhesive during use to deliver the medication. The base 12 can include an adhesive pad with a removable label commonly used in an infusion set for insulin injection as known in the art. The base 12 has a proximal end configured for coupling to the needle carrier 18. The base 12 includes an inner cavity 26 with a dimension for receiving the catheter hub 14 and allowing the catheter hub to move axially within the cavity as shown in FIGS. 3 and 4. In the embodiment shown, the distal open end 28 of the base 12 receives an end piece 30 for closing the cavity 26 and retaining the catheter hub 14 within the cavity. As shown in FIG. 2, the end piece 30 has a central opening 32 with a dimension for receiving the catheter 16 so that catheter extends through the end piece a distance for dispensing the medication or delivering the medication to the patient. The base 12 has a proximal end wall 56 enclosing the cavity 26 and having an opening 58 to receive the needle carrier and the insertion needle 20.

The catheter hub 14 supports the catheter 16 during use of the device for delivering the medication to the patient. The catheter 16 is shown as a flexible catheter commonly used in infusion sets, patch pumps and the like for insulin delivery. The catheter is made from a soft, flexible plastic material to provide a level of comfort to the patient when inserted into the patient for extended periods of time. The embodiments described herein refer to a catheter, such a flexible catheter although the delivery device can be a rigid cannula or catheter.

The catheter hub 14 in the embodiment shown has a distal surface 34 where the catheter extends from the distal surface. A proximal surface 36 is oriented opposite the distal surface 34. A side surface 38 extends between the distal surface 34 and the proximal surface 36. A coupling member 40 projects from the proximal surface 38 a distance for coupling to a delivery device. The coupling member 40 in the embodiment shown has substantially cylindrical shape with an annular groove 42 suitable for coupling with the delivery device and forms a fluid port for supplying the medication to the catheter. The needle hub and coupling member include a fluid flow passage for delivering the medication from the delivery device through the catheter and into the patient.

The needle carrier 18 in the embodiment shown supports the insertion needle 20 for inserting and positioning the catheter 16 into a surface, such as the skin of the patient. The needle carrier 18 as shown has a body 44 with a distal end 46 and a proximal end 48. The distal end 46 is configured for contacting the proximal surface 36 of the catheter hub 14. The body 44 has a dimension for holding by the user during the insertion of the catheter into the patient. In this embodiment, the needle 20 is coupled directly to the body 44.

The distal end 46 of the needle carrier 18 includes at least one and typically two coupling members 50 for coupling to the catheter hub 14 during use. The coupling member 50 includes a distally extending flexible arm 52 with an inwardly extending tab 54. The flexible arm 52 is formed with the body 44 and can flex outwardly with respect to the needle carrier 18. In the embodiment shown, the flexible arm 52 is configured to couple to the body 44 of the needle carrier 18 at a location spaced proximally from the distal end 46 and has a free end with the inwardly extending tab 54. In the initial ready position of the insertion device, the legs 52 extend through the opening 58 in the base for coupling with the catheter hub 14.

As shown in FIG. 3, the legs 52 have a length to extend past the distal end of the body 44 of the needle carrier 18 so that the legs are positioned in the cavity 26 of the body 22 of the base 12. The tabs 54 of the legs 52 are received in the annular groove 42 to couple the needle carrier to the catheter hub 14 and to the base 12. As shown in FIG. 3, the opening 58 in the end wall 56 has a dimension to receive the legs and prevent the legs from flexing outward so that the tabs of the legs remain coupled to the catheter hub 14.

In the ready position of the insertion device, the catheter 16 extends distally from the distal surface of the base with the insertion needle extending beyond the distal end of the catheter where the insertion needle can pierce the skin surface and position the catheter a first depth. During use of the insertion device, the insertion device in the initial ready position shown in FIG. 3 is pressed against the skin surface so that the insertion needle 20 and the catheter penetrate the skin surface. As shown in FIG. 3, the insertion force and drag on the skin caused by the insertion needle 20 and the catheter 16 form an indentation 60 in the skin surface 62 referred to as tenting of the skin surface. FIG. 3 shows the catheter and insertion needle at a first insertion depth with respect to the surface of the skin.

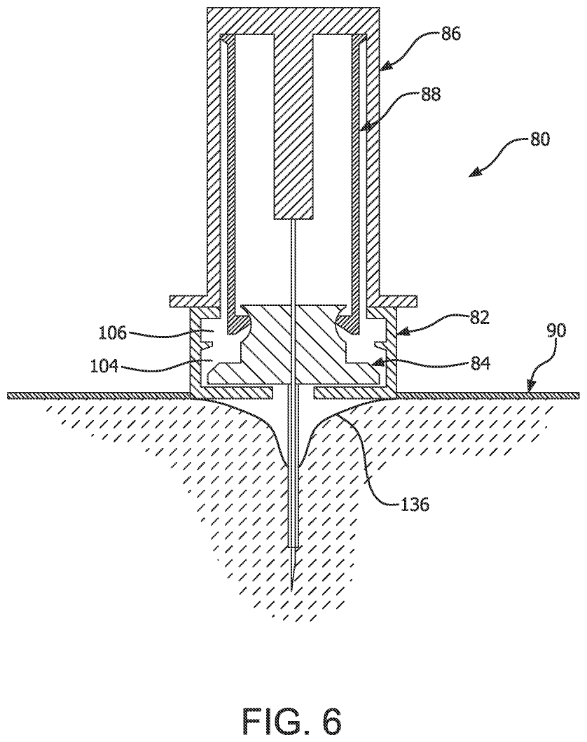

Referring to FIG. 4, needle carrier 18 can be retracted with respect to the base 12 to retract the catheter hub 14 from the initial position shown in FIG. 3 where the needle hub is in the distal portion of the cavity 16 to the proximal portion of the cavity as shown in FIG. 4. By retracting the needle carrier 18, the catheter hub 14, the catheter 16 and the insertion needle 20 are retracted to position shown in FIG. 4 so that the distal end of the catheter is at a second position less than the initial insertion position shown in FIG. 3. By retracting the catheter and insertion needle with respect to the skin surface, the depth of the indentation and tenting 60 shown in FIG. 6 is reduced or eliminated so the skin surface conforms substantially to the shape of the distal surface of the base 12.

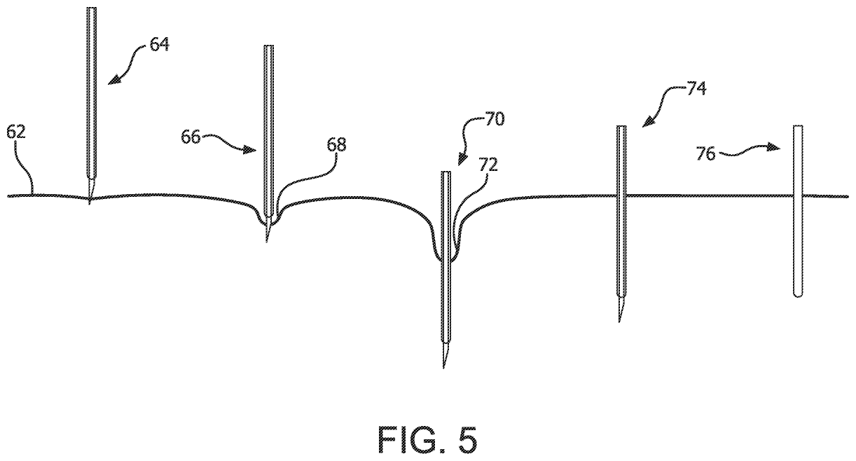

Referring to FIG. 5, the effect of the insertion force of the insertion needle and catheter is shown. As indicated by reference number 64, the insertion needle initially contacts the skin surface when the skin surface is substantially in the relaxed shape. Reference number 66 shows the initial deformation of the skin surface where tenting 68 of the skin surface begins. The deeper penetration of the insertion needle and catheter indicated by reference number 70 shows a deep indentation and tenting 72 and the distal end of the catheter a first depth relative to the skin surface. However, the tenting 72 and stretching of the skin surface inhibits the penetration of the distal end of the catheter to a desired depth for subcutaneous administration of the medication. The skin surface relaxing over time to the original shape can cause the distal end of the catheter to bend so as not to be positioned at the intended depth and can cause occlusion of the catheter.

As shown in FIG. 5 by reference number 74, retracting or withdrawing the catheter and the insertion needle a selected distance positions the distal end of the catheter at the desired depth in the skin surface and reduces or avoids the tenting of the skin surface. As indicated by reference number 76, the distal end of the catheter is now placed at the selected depth in the skin surface after the insertion needle is retracted. The skin surface is relaxed so minimal movement of the skin surface occurs which can reduce the possibility of occlusion of the catheter tip by the movement or relaxing of the skin surface after insertion and after the insertion needle is removed.

In another embodiment shown in FIGS. 6-8, the insertion device 80 is similar to the previous embodiment. The insertion device 80 includes a base 82, a catheter hub 84 received in the base 82, needle carrier 86, and a coupling member 88.

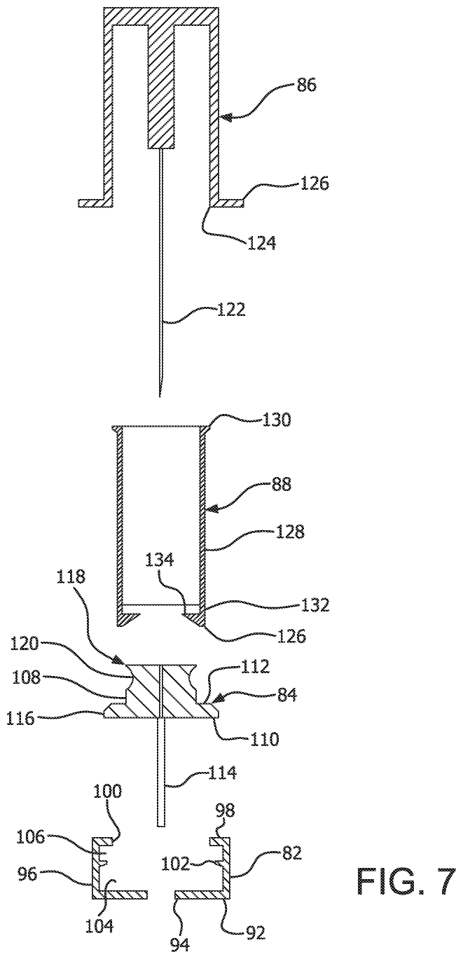

The base 82 as in the previous embodiment is configured for contacting the skin surface during the delivery of the medication. In the embodiment shown, an adhesive pad 90 is included on the distal face of the base 82 to attach the base to the skin surface during use. The base 82 having an end wall 92 with a distal surface are provided with an opening 94, a side wall 96, and a proximal wall 98 with an opening 100 as shown in FIG. 7. An internal flange 102 extends radially inward from the side wall 96 to form a first distal internal cavity 104 and a second proximal internal cavity 106.

The catheter hub 84 has a body 108 with a distal surface 110 and a proximal surface 112. A catheter 114 extends from the distal surface of the catheter hub 84. An outwardly extending flange 116 projects from the distal end of the body 108. The proximal end of the body 108 includes a fluid port 118 forming a coupling having an annular recess 120 for coupling with a fluid supply of a delivery device.

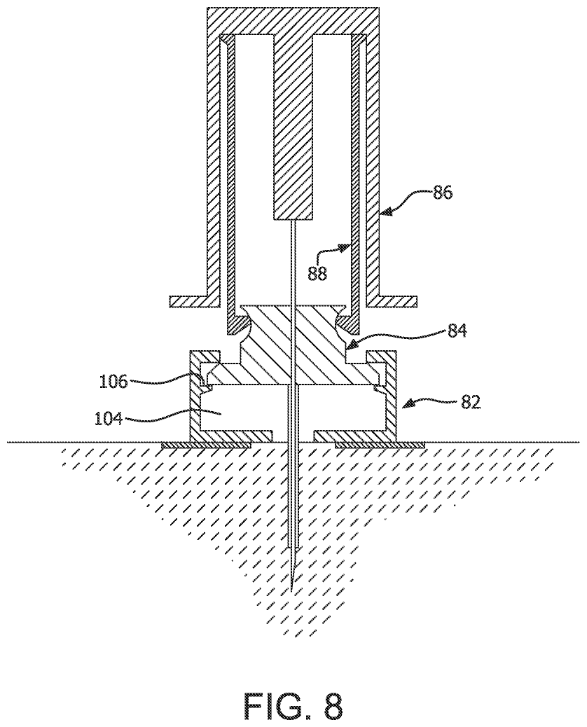

Referring to FIG. 8, the catheter hub 84 is received in the base 82 and is movable axially in the base between a distal position in the ready state of the insertion device to a proximal position after use. The flange 116 of the catheter hub 84 is positioned in the distal cavity 104 and retained between the distal end wall 92 and the internal flange 102. The catheter hub 84 can move to a proximal position by the flange 116 sliding over the flexible internal flange 102 so that the flange 116 is retained between the flange 102 and proximal end wall 98. As shown in FIG. 8, proximal end wall 98 of the base 82 has the opening 100 with a dimension to retain the flange 116 within the base 82 while allowing the coupling member 118 to extend through the opening 100.

The needle carrier 86 supports an insertion needle 122 that extends from a distal end 124 for extending through the catheter hub and the catheter. In the embodiment shown the needle carrier 86 has a shape complementing the proximal face of the base 82 with an outwardly extending flange 126 for contacting the base. The needle carrier 86 in the embodiment shown receives the coupling member 88 for axial movement with respect to the needle carrier. The coupling member 88 has outer shape and dimension to move and/or slide in the open distal end of the needle carrier. In the embodiment shown, the coupling member 88 has a side wall 128 with an open distal end 126 with a dimension to receive the port 118 of the catheter hub 84 and an open proximal end.

The side wall 128 of the coupling member 88 has an outwardly extending detent 130 at the proximal end for engaging the inner surface of the needle carrier 86. The distal end 124 of the needle carrier 86 has at least two flexible legs 132 with an inwardly extending tab 134. The legs 132 extend in a distal direction a distance for coupling with the fluid port 118. The tabs 134 have a dimension to be received in the annular recess 120 of the port 118.

The insertion device 80 is assembled in the ready position as shown in FIG. 6 where the catheter hub 84 is in the distal position in the base 82 and the insertion needle 122 extending through the catheter 114. In the position shown in FIG. 6, the catheter and insertion needle project from the base a first distance. In the ready position shown in FIG. 6, the insertion needle and catheter can be inserted into the skin surface to a first depth, which can cause tenting indicated by reference number 136. As shown in FIG. 8, the needle carrier 86 is retracted which carries the insertion needle 122 and the coupling member 88 in a proximal direction to move the catheter hub 84 from the first distal cavity 104 past the flange 102 and into the second proximal cavity 106. By retracting the insertion needle and the catheter, the tenting of the skin surface is avoided as shown in FIG. 8. The needle carrier 86 can be retracted so that the coupling member 88 slides from the open end of the needle carrier where the legs 132 are free from the side wall of the needle carrier. The legs 132 can then flex outwardly by the retracting force to separate the legs from the catheter hub. The catheter hub can then be connected to a fluid supply for delivering the medication to the patient through the catheter.

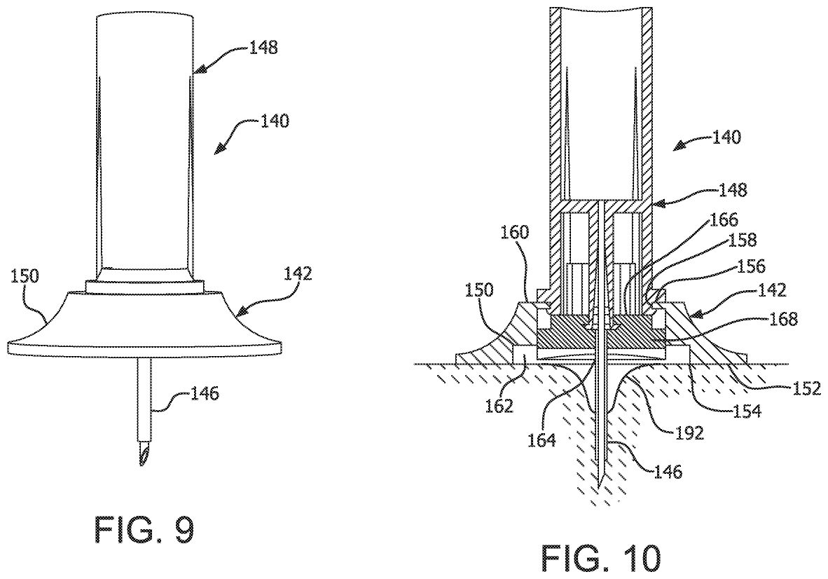

In another embodiment shown in FIGS. 9-12, the insertion device 140 is similar to the previous embodiment and includes a base 142, a catheter hub 144 supporting a catheter 146, and needle carrier 148. The base 142 as in the previous embodiment, has a body 150 with a distal face 152 having an opening 154, a proximal end wall 156 with an opening 158 forming an internal cavity 160. An end piece 162 is received in the opening 158 to enclose the cavity 160. The end piece includes an opening 164 for the catheter 146.

The catheter hub 144 has a shape and configuration for moving and/or sliding in the cavity 160 of the base. The catheter hub 144 has a proximal end 166 with a dimension for sliding through the opening in the end wall 156 and a body portion 168 with a dimension to slide within the cavity and be retained by the end wall. As in the previous embodiment, the catheter hub is able to slide axially within the cavity from a distal ready position to a retracted position. The catheter hub has an axial bore for receiving the catheter 146. The end portion 166 has a recess 170 around the axial bore at the proximal end of the catheter as shown in FIGS. 10-12. The recess 170 has an inwardly extending detent 172 shown in FIG. 12.

The needle carrier 148 has a body 174 with a centrally located support for supporting an insertion needle 176 and an outer wall 178. The distal end of the outer wall 178 has an outwardly extending detent 180 and a flange 182 spaced proximally from the detent a distance corresponding to a thickness of the end wall 156.

A coupling member 184 extends distally from the needle carrier 148 and is configured for coupling with the catheter hub 144. In the embodiment shown in FIG. 10, the coupling member 184 includes a pair of flexible legs 186 extending distally from the post 188 on opposite sides of the insertion needle 176. The flexible legs 186 include an outwardly projecting tab 190 and are biased radially outward as shown in FIG. 10 for coupling with the detents 172 in the recess 170 of the catheter hub 144.

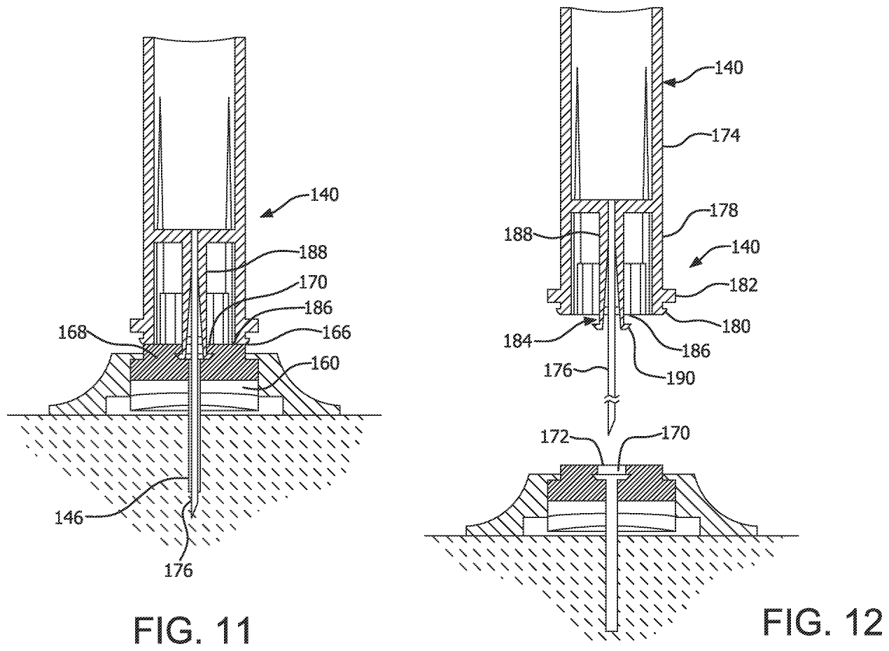

The insertion device 140 in the ready position shown in FIG. 10 has the needle carrier 148 coupled to the base 142 by the detent 180 and the flange 182. The catheter hub 144 is oriented in the distal position within the cavity 160 by the coupling member 184 positioning the catheter hub relative to the needle carrier. The insertion device 140 is forced against the skin surface as shown in FIG. 10 so that the insertion needle 176 and the catheter 146 pierce the skin surface and the catheter 146 pierces the skin surface to a first depth that can cause tenting 192 of the skin surface. After the catheter and insertion needle pierce the skin surface to depth shown in FIG. 10, the needle carrier 148 can be separated from the base 142 by a withdrawing force. The withdrawing or retracting movement of the needle carrier and the needle, move the catheter hub to the position shown in FIG. 11 to reduce the tenting of the skin surface and withdraw the distal end of the catheter to a second depth less than the depth of the initial insertion. Further axial movement of the needle carrier causes the legs 186 of the coupling member 184 to flex inwardly and separate from the catheter hub 144 and the base 142. The proximal end of the base and catheter hub can then be connected to a suitable fluid supply to deliver the medication to the patient through the catheter.

The embodiments of the insertion device provide a mechanism for reducing tenting of the skin surface around the insertion site of the catheter and the insertion needle. A method of deploying a catheter and introducing a catheter into a skin surface with reduced tenting of the skin surface by the insertion of the catheter and insertion needle can be obtained by the embodiments of the insertion device. As shown in the embodiments, a catheter and insertion needle are introduced into the skin surface to a first depth with respect to the surface of the skin. The catheter and insertion needle are retracted relative to the base of the insertion device and the skin surface to a second position with respect to the base where the second position is a retracted position with respect to the first position. Retracting the insertion needle and the catheter to the second position reduces the tenting at the skin surface around the insertion site.

While various embodiments have been shown and described, it will be understood by those skilled in the art that various changes and modifications can be made without departing from the scope of the invention as defined in the appended claims.

1. A catheter insertion device comprising:
a base configured for contacting a surface;
a catheter hub movable relative to said base;
a catheter coupled to said catheter hub and extending from a distal side of said catheter hub and extending from a distal side of said base;
a needle carrier having an insertion needle within said catheter, said needle carrier coupled to said catheter hub and movable with respect to said base, said needle carrier configured to position the catheter hub in a first position with respect said base during insertion of said catheter into the surface and to move the catheter hub and catheter to a second position with respect to said base when said insertion needle is retracted with respect to said base.

2. The catheter insertion device of claim 1, wherein said needle carrier has a coupling mechanism for coupling to said catheter hub when said catheter hub is in said first position and separates from said catheter hub when said catheter hub is in said second position.

3. The catheter insertion device of claim 1, wherein said coupling mechanism of said needle carrier includes at least two flexible legs extending distally from said needle carrier and being removably coupled to said catheter hub.

4. The catheter insertion device of claim 3, wherein said at least two flexible legs are removably coupled to an outer surface of said catheter hub.

5. The catheter insertion device of claim 3, wherein said catheter hub has a proximal side with a central recess, and where said at least two flexible legs are removably coupled to an inner surface of said recess.

6. The catheter insertion device of claim 5, wherein said recess has at least one inwardly extending detent and said at least two flexible legs have an outwardly extending coupling tab for removably coupling with said detent.

7. The catheter insertion device of claim 1, wherein said base has an inner cavity and said catheter hub moves axially within said base, said first position of said catheter hub is a distal position where said catheter extends from said base a first distance, and said second position of said catheter hub is a proximal position and where said catheter extends from said base a second distance less than said first distance.

8. The catheter insertion device of claim 7, wherein said catheter hub has proximal portion positioned in said inner cavity when said catheter hub is in said distal position, and said proximal portion projects from said base when said catheter hub is in said proximal position.

9. The catheter insertion device of claim 8, wherein said proximal portion of said catheter hub is a fluid port for supplying a medication to said catheter.

10. The catheter insertion device of claim 1, wherein said base has an internal cavity receiving said catheter hub, said internal cavity has an internal flange defining a distal space and a proximal space, and where said catheter hub is received in said distal space when said catheter hub is in the first position and the catheter hub is received in the proximal space when the catheter hub is in the second position.

11. A catheter insertion device comprising:
a base configured for contacting a surface, said base having an internal cavity with an open distal surface and an open proximal surface;
a catheter hub received in said internal cavity and movable relative to said base from a distal position to a proximal position;
a catheter coupled to said catheter hub and extending from a distal side of said catheter hub and extending from a distal side of said base a first distance when said catheter hub is in said first position and extending from said base a second distance less than said first distance when said catheter hub is in said proximal position; and
a needle carrier having an insertion needle within said catheter, said needle carrier coupled to said catheter hub and movable with respect to said base, said needle carrier configured to position the catheter hub in the distal position with respect said base during insertion of said catheter into the surface and to move the catheter hub and catheter to the proximal position with respect to said base when said insertion needle is retracted with respect to said base.

12. The catheter insertion device of claim 11, wherein said needle carrier has a coupling mechanism for coupling to said catheter hub when said catheter hub is in said first position and separates from said catheter hub when said catheter hub is in said second position.

13. The device of claim 12, wherein said coupling mechanism of said needle carrier includes at least two flexible legs extending distally from said needle carrier and being removably coupled to an outer surface of said catheter hub.

14. The device of claim 12, wherein said coupling mechanism of said needle carrier includes at least two flexible legs extending distally from said needle carrier and being removably coupled to an inner surface of said catheter hub.

15. A method of deploying a catheter into a surface, said method comprising the steps of:
introducing a catheter and an insertion needle of an insertion device into a surface to a depth where a distal end of said catheter is spaced a first distance from a base of the insertion device;
retracting the catheter and insertion needle with respect the base of the insertion device to a second distance less than the first distance to reduce tenting of the surface at the insertion site, and retracting the insertion needle from the catheter and catheter hub.

16. The method of claim 15, wherein said insertion device includes a needle carrier removably coupled to a catheter hub supporting the catheter and where the catheter hub is movable within the base from a distal position to a proximal position, said method including the step of retracting the needle carrier to move the catheter hub to the proximal position and to retract the catheter with respect to the insertion device.

17. The method of claim 16, further comprising separating said needle carrier from said catheter hub when said catheter hub is in said proximal position.

18. The method of claim 17, comprising inserting said catheter and insertion needle into the skin surface when said catheter hub is in said distal position, and moving said catheter hub to said proximal position after the insertion.

19. The method of claim 18, wherein the needle carrier includes at least two flexible legs removably coupled to the catheter hub, said method comprising separating the flexible legs from the catheter hub by retracting the needle carrier with respect to the insertion device.

20. The method of claim 19, further comprising the step of connecting the catheter to a fluid supply when the catheter hub is in the proximal position.
